(19) United States
(12) Patent Application Publication
(10) Pub. No.: US 2016/0318992 A1
(43) Pub. Date: Nov. 3, 2016

(54) METHODS FOR DEPYROGENATION OF PROTEINS

(71) Applicant: NOVABONE PRODUCTS, LLC, Alachua, FL (US)

(72) Inventors: Gregory J. Pomrink, Newberry, FL (US); Chloë Goldbach, Gainesville, FL (US); Zehra Tosun, Gainesville, FL (US)

(21) Appl. No.: 15/140,104

(22) Filed: Apr. 27, 2016

Related U.S. Application Data
(60) Provisional application No. 62/153,851, filed on Apr. 28, 2015.

(51) Int. Cl. C07K 14/78; A61L 27/24; A61K 38/39; A61K 33/00; A61K 45/06

(52) U.S. Cl. CPC C07K 14/78; A61K 33/00; A61K 45/06; A61K 38/39; A61L 27/24; A61L 2430/02

(57) ABSTRACT

Methods relating to depyrogenaton of proteins including vapor hydrogen peroxide treatment, gaseous chloride dioxide treatment or dehydrothermal treatment are described.

### CROSS-REFERENCE TO RELATED APPLICATIONS

This application claims the benefit of U.S. Provisional Patent Application No. 62/153,851, filed Apr. 28, 2015, the entire contents of which are hereby incorporated herein by reference.

### BACKGROUND

Methods of preparing pyrogen-free proteins, such as collagen, adapted for use in the manufacture of protein derived products, such as medical devices or other products intended for medical uses, particularly in surgical procedures are described.

Potential sources of contamination during production of medical products include the raw materials, equipment and processes during production, in addition to the facility and personnel (Kushwaha P, Microbial Contamination, A regulatory Perspective Journal of Pharmacy Research, 3(1): 124-31 (2010)).

In the preparation of a wide variety of protein-derived, such as collagen-derived products for medical uses, it is necessary that the product be free of microorganisms such as bacteria, yeasts, molds and the like. These microorganisms may be destroyed or rendered innocuous readily, for example by sterilization by subjecting the collagen source material and/or collagen derived product to radiation, bactericides, moldicides, various gases, and heat treatment.

Pyrogens, on the other hand, are not living organisms and are not rendered innocuous by bactericides, moldicides and gases and are thermostable. Pyrogens are generally considered to be thermostable products of the growth of strains of bacteria, yeasts and molds, some being soluble and others being insoluble and filterable. In addition to their fever producing affects, pyrogens have physiologic effects on the circulatory system, the endocrine glands and metabolic processes. The rise in body temperature is only one of the manifestations to the introduction of minute quantities of pyrogenetic substances into the body and the specific effects will be dependent upon the individual subject. While the microorganisms may be rendered innocuous by a sterilization treatment of the final product, it is also essential that pyrogenetic substances be removed from the product. While, thermal, UV light and ethylene oxide treatments or gamma and electron beam irradiation may reduce pyrogen levels, concerns have been raised regarding the adverse effects that these techniques may have on protein. Specifically, these techniques are known to crosslink, denature, or change the tertiary structures of the protein.

Ultrafiltration, which is a type or variation of membrane filtration in which forces like pressure or concentration gradients lead to a separation through a semipermeable membrane, is one of the known methods used for protein depyrogenation. Suspended solids and solutes of high molecular weight are retained in the so-called retentate, while water and low molecular weight solutes pass through the membrane in the permeate. This separation process is used in industry and research for purifying and concentrating macromolecular (10³-10⁶ Da) solutions, especially protein solutions. However, it is a very slow and expensive process and often used for small scale filtration methods. Moreover, ultrafiltration, along with chromatography and distillation methods may result in protein structure alterations.

As such, there exists a need for new and or improved methods of protein depyrogenation that are efficient, inexpensive, effective, and do not result in denaturing of the protein.

### SUMMARY

Certain embodiments relate to a method for depyrogenaton of protein including exposing the protein to vapor hydrogen peroxide (VHP) for a duration of time and at a concentration of vapor hydrogen peroxide sufficient to reduce pyrogens of protein, wherein the exposing step does not substantially change the tertiary structure of the protein and/or does not denature the protein. In the VHP method, the concentration of hydrogen peroxide may be in a range from about 200 ppm to about 2000 ppm hydrogen peroxide in an atmospheric pressure isolator, and the duration of the exposing step may range from about 1 hours to about 48 hours. In the method, the concentration of hydrogen peroxide may be about 800 ppm hydrogen peroxide in an atmospheric pressure isolator, and the duration of the exposing step may be about 4 hours. The method may further comprise aerating the protein. The protein may be selected from the group consisting of and not limited to collagen, fibronectin, vitronectin, laminin, pectin, elastin, osteopontin, bone sialoprotein, thrornbospondin, and fibrinogen, gelatin, and combinations thereof.

Certain further embodiments relate to a method for depyrogenaton of protein, the method including exposing the protein to gaseous chloride dioxide for a duration of time and at a concentration of gaseous chloride dioxide sufficient to reduce pyrogens of the protein, wherein the exposing step does not substantially change the tertiary structure of the protein and/or does not denature the protein. In the method the concentration of gaseous chloride dioxide may be in a range from about 100 ppm to about 2000 ppm gaseous chloride dioxide per hour in an atmospheric pressure isolator. In the method, the concentration of gaseous chloride dioxide may be about 720 ppm gaseous chloride dioxide per hour in an atmospheric pressure isolator. The protein may be selected from the group consisting of and not limited to collagen, fibronectin, vitronectin, laminin, pectin, elastin, osteopontin, bone sialoprotein, thrombospondin, and fibrinogen, gelatin, and combinations thereof.

Other embodiments relate to a method for depyrogenaton of protein including exposing the protein to a dehydrothermal treatment (DHT) for a duration of time sufficient to reduce pyrogens, wherein the exposing step does not substantially change the tertiary structure of the protein and/or does not denature the protein. In the method, the exposing step may be at a temperature ranging from about 60° C. to about 130° C. and under a pressure of from about 10 mTorr to about 1000 mTorr. Alternatively, the exposing step may be at a temperature of about 105° C. and under a pressure of 150 mTorr. In the method, the duration of time sufficient to reduce pyrogens may be from about 1 hour to 48 hours. The protein may be collagen, fibronectin, vitronectin, laminin, pectin, elastin, osteopontin, bone sialoprotein, thrombospondin, and fibrinogen, gelatin, and combinations thereof.

A further embodiment relates to a composition comprising collagen with substantially no amount of pyrogens, substantially no change in the tertiary structure of collagen, and substantially no denaturing of collagen.

### BRIEF DESCRIPTION OF THE DRAWINGS

FIG. 1 depicts a graph illustrating the conditions with the highest reduction levels (DHT, VHP conditions TS3 and TS4, and the ClO₂ condition) compared to the raw collagen pyrogen levels.

FIG. 2 shows a graph depicting a DSC scan for raw collagen treatment sample.

FIG. 3 shows a graph depicting a DSC scan for raw collagen treatment sample.

FIG. 4 shows a graph depicting a DSC scan for the 3 hour DHT collagen treatment sample.

FIG. 5 shows a graph depicting a DSC scan for the 24 hour DHT collagen treatment sample.

FIG. 6 shows a graph depicting a DSC scan for TS3 VHP treated collagen sample.

FIG. 7 shows a graph depicting a DSC scan for TS4 VHP treated collagen sample.

FIG. 8 shows a graph depicting a DSC scan for ClO₂ treated collagen sample.

FIG. 9 shows the Dunnett's test results.

FIG. 10 displays a graphical comparison of each of the percent collagen structures for each of the five digested collagen samples.

### DETAILED DESCRIPTION OF THE DRAWINGS AND THE PRESENTLY PREFERRED EMBODIMENTS

The following detailed description illustrates the invention by way of example, not by way of limitation of the scope, equivalents or principles of the invention. This description will clearly enable one skilled in the art to make and use the invention, and describes several embodiments, adaptations, variations, alternatives and uses of the invention, including what is presently believed to be the best modes of carrying out the invention.

In this regard, the invention is illustrated in the several figures, and is of sufficient complexity that the many parts, interrelationships, and sub-combinations thereof simply cannot be fully illustrated in a single patent-type drawing. For clarity and conciseness, several of the drawings show in schematic, or omit, parts that are not essential in that drawing to a description of a particular feature, aspect or principle of the invention being disclosed. Thus, the best mode embodiment of one feature may be shown in one drawing, and the best mode of another feature will be called out in another drawing.

All publications, patents and applications cited in this specification are herein incorporated by reference as if each individual publication, patent or application had been expressly stated to be incorporated by reference.

Described are methods for depyrogenating proteins, such as collagen. The safety of utilizing proteins in medical type applications, including medical devices, is directly linked to protein pyrogen levels. As such, effective and non-toxic methods of removing pyrogens from the protein without cross-linking or denaturing the protein are advantageous.

The proposed methods include vapor hydrogen peroxide (VHP), chlorine dioxide, and dehydrothermal treatments (DHT) to depyrogenate proteins, such as collagen. Advantageous methods of depyrogenation using VHP, chloride dioxide and DHT do not change the tertiary structures of the proteins and/or do not denature the proteins. Furthermore, VHP and chloride dioxide methods do result in crosslinking of the proteins. Following pyrogen level reduction from implementing one of these methods, the protein can be used in medical devices and medical applications.

The protein may be any protein, including but not limited to collagen, fibronectin, vitronectin, laminin, pectin, elastin, osteopontin, bone sialoprotein, thrombospondin, and fibrinogen, and combinations thereof. The protein may be any insoluble protein.

Certain embodiments relate to a composition comprising collagen with substantially no amount of pyrogens, substantially no change in the tertiary structure of collagen, and substantially no denaturing of the collagen.

The term “depyrogenation” refers to the removal of pyrogens from a material, most commonly from implantable devices or products, injectable pharmaceuticals, etc. A “pyrogen” is defined as any substance that can cause a fever. Bacterial pyrogens include endotoxins and exotoxins, although many pyrogens are endogenous to the host. Endotoxins include lipopolysaccharide (LPS) molecules found as part of the cell wall of Gram-negative bacteria, and are released upon bacterial cell lysis. Endotoxins may become pyrogenic when released into the bloodstream or other tissue where they are not usually found.

The term “a duration of time sufficient to reduce pyrogens” refers to a time period sufficient to reduce at least 0% of pyrogens; at least 25% of pyrogens; at least 50% pyrogens; at least 75% of pyrogens; at least 90% of pyrogens; at least 95% of pyrogens; and, preferably at least 99.99% of pyrogens present in a protein or protein containing product. The duration of time sufficient to reduce pyrogens may be, for example, at least 1 hour, at least 2 hours, at least 3 hours, at least 4 hours, at least 6 hours, at least 12 hours, at least 24 hours, or at least 48 hours. The duration of time sufficient to reduce pyrogens may also be 1 pulse, 2 pulses, or 3 or more pulses depending on the type of equipment or method of depyrogenaton used.

The terms “reduce” or “reduced” refer to a decrease or reduction in the amount of pyrogens present following a specific treatment method to remove pyrogens as compared to the amount of pyrogens present in the absence of treatment. Desirably a degree of decrease is greater than 10%, 25%, 50%, 75%, 90%, 95% or 99.99% as compared to the amount of pyrogens in the absence of treatment.

The term “substantially” means essentially the same or similar.

The terms “does not substantially change” or “substantially no change” in connection with the tertiary structure of the protein mean that the tertiary structure of the protein following the depyrogenation treatment will remain similar to the tertiary structure of the protein before the depyrogenation treatment. For example, if the pre-treatment sample consisted of 20% unorganized protein, the post treatment sample would consist of <30% unorganized protein.

The term “tertiary structure” of the protein refers to the three-dimensional structure of a protein or protein's geometric shape. The tertiary structure will have a single polypeptide chain “backbone” with one or more protein secondary structures, the protein domains. Amino acid side chains may interact and bond in a number of ways. The interactions and bonds of side chains within a particular protein determine its tertiary structure. The protein tertiary structure is defined by its atomic coordinates. These coordinates may refer either to a protein domain or to the entire tertiary structure (Kyte, J. “Structure in Protein Chemistry.” Garland Publishing, New York. 1995. ISBN 0-8153-1701-8). A number of tertiary structures may fold into a quaternary structure.

### Vapor Hydrogen Peroxide (VHP)

One embodiment of the present invention relates to a method for depyrogenaton of protein, the method including exposing the protein to VHP for a duration of time and at a concentration of VHP sufficient to reduce pyrogens (“biodecontamination” step), wherein the exposing step does not result in cross-linking or denaturing of the protein.

The concentration of hydrogen peroxide may be in a range from about 600 ppm to about 1000 ppm hydrogen peroxide in an atmospheric pressure isolator, and the duration of the exposing step ranges from about 2 hours to about 6 hours. In a preferred embodiment, the concentration of hydrogen peroxide may be about 800 ppm hydrogen peroxide in an atmospheric pressure isolator, and the duration of the exposing step is about 4 hours.

In certain embodiments, the method further includes dehumidification, conditioning and aeration steps.

The protein may be any protein, including but not limited to collagen, fibronectin, vitronectin, laminin, pectin, elastin, osteopontin, bone sialoprotein, thrombospondin, and fibrinogen, gelatin, and combinations thereof. The protein may be any insoluble protein.

The method using vapor hydrogen peroxide can be implemented for protein depyrogenation (e.g., collagen) using, e.g., the Steris MD2000 deep vacuum sterilization system and the Steris VHPI000ED VHP generator with 30 ft³ isolator.

During a cycle, humidity may be first removed using an integrated desiccant system followed by rapid injection of Vaprox (VHP sterilant) to condition the system and quickly raise the hydrogen peroxide level to a desired concentration. The VHP can be maintained at the desired concentration for a set amount of time for the biodecontamination step and finally the vapor is broken down into safe byproducts, water vapor and oxygen, once the treatment process has been completed.

### Chloride Dioxide

Chlorine dioxide is known to be a disinfectant, as well as a strong oxidizing agent. The bactericidal, algaecidal, fungicidal, bleaching, and deodorizing properties of chlorine dioxide are also well known. Therapeutic and cosmetic applications for chlorine dioxide are known.

Certain embodiments relate to a method for depyrogenation of protein, such as collagen including exposing the protein to gaseous chloride dioxide for a duration of time and at a concentration of gaseous chloride dioxide sufficient to reduce pyrogens (“biodecontamination” step), wherein the exposing step does not result in cross-linking or denaturing of the protein.

In certain embodiments, a commercially available chlorine dioxide system from ClorDiSys, including but not limited to Minidox, Megadox, Steridox, and Cloridox systems, may be used for collagen depyrogenation using gaseous chlorine dioxide. Specifically, the gaseous chlorine dioxide method for depyrogenation can be performed by ClorDiSys for collagen depyrogenation using an enclosed chlorine dioxide chamber at a dosage of 720 ppm per hour.

In certain embodiments, the collagen samples can be depyrogenated within Tyvek pouches provided by ClorDiSys. Collagen samples exhibit a pink coloration following treatment to indicate exposure to the chlorine dioxide treatment process. The chlorine dioxide treatment process is not affected by temperature, produces no measurable residue, is non-carcinogenic, is able to kill all viruses, bacteria, fungi and spores, and is able to completely fill all space contained in the chamber in order to evenly contact all surfaces.

### Dehydrothermal Treatment (DHT)

Certain further embodiments relate to a method for depyrogenaton of protein, such as collagen, including exposing the protein to dehydrothermal treatment for a duration of time sufficient to reduce pyrogens, wherein the exposing step does not result in denaturing of the protein.

DHT removes water from collagen and the resulting condensation reactions have the potential to crosslink the collagen molecules. The heat treatment provided by DHT removes pyrogens in addition to water molecules.

In certain embodiments, the exposing step may be at a temperature ranging from about 40° C. to about 200° C. and under a pressure of from about 10 mTorr to about 1000 mTorr. Alternatively, the exposing step may be at a temperature of about 105° C. and under a pressure of 150 mTorr.

In certain further embodiment, the duration of time sufficient to reduce pyrogens may be from about 1 hour to 48 hours (e.g., 3, 6, 12, and 24 hours). The protein may be and is not limited to collagen, fibronectin, vitronectin, laminin, pectin, elastin, osteopontin, bone sialoprotein, thrombospondin, fibrinogen, gelatin, or combinations thereof.

### Compositions

Any protein, such as collagen, depyrogenated according to the described methods with substantially no amount of pyrogens, substantially no change in the tertiary structure of collagen, and substantially no denaturing of collagen may be included in a composition. For example, certain embodiments relate to a composition comprising collagen with substantially no amount of pyrogens, substantially no change in the tertiary structure of collagen, and substantially no denaturing of collagen.

The composition may be suitable for wound care, hemostasis, duraplasty, as an adhesion barrier or for use in other medical applications.

In certain embodiments, the composition may include a ceramic material, such as bioactive glass, tricalcium phosphate (TCP), hydroxyapatite calcium sulfate, or the like.

Bioactive glass may be melt-derived or sol-gel derived. Depending on their composition, bioactive glasses of the invention may bind to soft tissues, hard tissues, or both soft and hard tissues. The composition of the bioactive glass may be adjusted to modulate the degree of bioactivity. Furthermore, borate may be added to or substituted for silica in the bioactive glass to control the rate of degradation. Additional elements, such as copper, zinc, silver and strontium may be added to bioactive glass to facilitate healthy bone growth. Bioactive glass that may also be suitable include glasses having about 40 to about 60 wt % SiO₂, about 10 to about 34 wt % Na₂O, up to about 20 wt % K₂O, up to about 5 wt % MgO, about 10 to about 35 wt % CaO, 0 to about 35 wt % SrO, up to about 50 wt % B₂O₃, and/or about 0.5 to about 12 wt % P₂O₅. The bioactive glass may additionally contain up to 10 wt % CaF₂.

A bioactive glass suitable for the present compositions may have silica, sodium, calcium, strontium, phosphorous, and boron present, as well as combinations thereof. In some embodiments, sodium, boron, strontium, and calcium may each be present in the compositions in an amount of about 1% to about 99%, based on the weight of the bioactive glass. In further embodiments, sodium, boron, strontium and calcium may each be present in the composition in about 1%, about 2%, about 3%, about 4%, about 5%, about 6%, about 7%, about 8%, about 9%, or about 10%. In certain embodiments, silica, sodium, boron, and calcium may each be present in the composition in about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90 to about 95%, or about 95 to about 99%. Some embodiments may contain substantially one or two of sodium, calcium, magnesium, strontium, and boron with only traces of the other(s). The term “about” as it relates to the amount of calcium phosphate present in the composition means+/−0.5%. Thus, about 5% means 5+/−0.5%.

The bioactive glass may further comprise one or more of a silicate, borosilicate, borate, strontium, or calcium, including SrO, CaO, P₂O₅, SiO₂, and B₂O₃. An exemplary bioactive glass is 4555®, which includes 46.1 mol % SiO₂, 26.9 mol % CaO, 24.4 mol % Na₂O and 2.5 mol % P₂O₅. An exemplary borate bioactive glass is 45S5B1, in which the SiO₂ of 45S5 bioactive glass is replaced by B₂O₃. Other exemplary bioactive glasses include 58S, which includes 60 mol % SiO₂, 36 mol % CaO and 4 mol % P₂O₅, and S70C30, which includes 70 mol % SiO₂ and 30 mol % CaO. In any of these or other bioactive glass materials described herein, SrO may be substituted for CaO.

The following composition, having a weight % of each element in oxide form in the range indicated, will provide one of several bioactive glass compositions that may be used to form a bioactive glass:

| | |
|---|---|
| SiO₂ | 0-86 |
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-25 |
| NaF | 0-35 |

Some examples of bioactive glass include silicate bioactive glass, a borate bioactive glass, titanate bioactive glass, and zirconate bioactive glass. The bioactive glass is melt-derived or sol-gel derived.

Furthermore, in certain embodiments, metallic materials, such as gold, silver, platinum, copper, palladium, iridium, strontium, cerium, or isotopes, or alloys, or salts thereof, may be incorporated (e.g., either by coating the surface of the bone grafting composition or by including or integrating the metallic materials in the structure of the bone grafting composition) into the described composition. These materials are able to conduct an electrical current and prevent or reduce body's inflammatory response at or near the injury site upon the delivery of the composition comprising a metallic material, enhancing the activity of, e.g., the calcium salt and the bone healing process. When bone is injured, it generates an electrical field. This low-level electrical field is part of the body's natural process that stimulates bone healing. When this healing process fails to occur naturally, a conductive implant material can facilitate regeneration of the bone. Conductive implants provide a safe, treatment that helps promote healing in fractured bones and spinal fusions which may have not healed or have difficulty healing. The devices stimulate the bone's natural healing process by sending low-level pulses of electromagnetic energy to the injury or fusion site. Importantly, electrical conductance and reduction of inflammation at the site of a wound may increase the rate at which the wound heals. Metallic materials may also promote wound healing by initiating or promoting angiogenesis. Increased blood flow may increase the rate of wound healing. Other benefits of gold may also be present. The term “metallic material” refers to pure metals, such as gold, silver, platinum, copper, palladium, iridium, strontium, cerium or isotopes (including radioisotopes), or alloys, or salts (the ionic chemical compounds of metals) thereof or other metallic materials having an atomic mass greater than about 45 and less than about 205. The term “atomic mass” is the mass of an atomic particle, sub-atomic particle, or molecule. It is commonly expressed in unified atomic mass units (u) where by international agreement, 1 unified atomic mass unit is defined as 1/12 of the mass of a single carbon-12 atom (at rest). The metallic material may be present in approximate amounts of 0.001-20 wt. % ratio with reference to the total weight of the composition. Alternatively, the metallic material may be present in approximate amounts of 0.001-10 wt. % ratio with reference to the total weight of the composition. The metallic material may also be present in a weight ratio of less than 10 wt. %; less than about 5 wt. %; less than about 2.5 wt. %; less than about 1 wt. %; or less than about 0.5 wt. %. In some embodiments, the weight ratio may be about 0.1%, about 0.2%, about 0.3%, about 0.4%, about 0.5%, about 0.6%, about 0.7%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.5%, about 4%, about 4.5%, or about 5%.

The compositions comprising depyrogenated proteins according to the described methods may be further combined (before, during or after the treatments) with a bioactive agent. The bioactive agent comprises one of antibodies, antigens, antibiotics, wound sterilization substances, thrombin, blood clotting factors, conventional chemo- and radiation therapeutic drugs, VEGF, antitumor agents such as angiostatin, endostatin, biological response modifiers, and various combinations thereof.

In certain embodiments, the compositions comprising depyrogenated proteins according to the described methods may be further combined (before, during or after the treatments) with polymers to provide further structural support. For example, bioactive glass and collagen composition may be prepared that further includes a block copolymer of ethylene oxide and propylene oxide.

In certain further embodiments, the compositions comprising depyrogenated proteins according to the described methods may be further combined (before, during or after the treatments) with glycosaminoglycans. U.S. Pub. No. 2014-0079789A1 to Pomrink et al., which is incorporated herein in its entirety, provides examples of bioactive glass-ceramics with glycosaminoglycans (GAGs). GAGs are polysaccharides that are present in various cells. There are many different types of GAGs have been found in tissues and fluids of humans, animals, and other vertebrates. GAGs are typically linear molecules with greatly varying chain lengths composed of heterogeneous polysaccharides and are formed by long disaccharide units with varying degrees of linkage, acetylation, and sulfation. The disaccharide units include galactose, N-acetylglucosamine, N-acetylgalactosamine, and glucuronic add. GAGs are often classified as being sulfated or non-sulfated, Known GAGs have been classified as being one of chondroitin sulfate, keratan sulfate, dermatan sulfate, hyaluronic acid, heparin, and heparan sulfate. Along with collagen, GAGs provide significant structural support to animal tissue. Without GAGs, tissues would not undergo proper repair. Further, the protection and maintenance of all tissues depends upon GAGs. Thus. GAGs can serve to provide further support to wounded tissue, particularly in the context of wounded tissue at or near the site of a bone injury.

In the compositions comprising depyrogenated proteins processed according to the described methods, the bioactive glass may be in a form of particles, spheres, fibers, mesh, sheets or a combination of these forms i.e. fibers within a sphere. The composition, porosity and particle sizes of the bioactive glass may vary.

The bioactive glass may vary in size. For example, the particles of the bioactive glass may range in size from 0.01 μm to 5 mm. Other ranges include about 1-5 micrometers, about 5-15 micrometers, about 15-50 micrometers, about 50-200 micrometers, about 200-1,000 micrometers, about 1-2 millimeters, about 2-3 millimeters, about 3-4 millimeters, or about 4-5 millimeters. In some embodiments, the bioactive glass particle has a diameter of between about 0.01 micrometer and about 5,000 micrometers.

In certain embodiments, the bioactive glass may include 0-80%<100 μm, 0-80%<500 μm, 0-80% 500-1000 μm, 0-80% 1000-2000 μm, 0-80% 2000-5000 μm, 0-90% 90-710 μm, and 0-90% 32-125 μm bioactive glass.

In certain embodiments, the compositions including depyrogenated proteins processed according to the described methods may be for use in regenerating bone at or near the site of a bony defect.

### EXAMPLES

The purpose of the testing was to evaluate various methods for depyrogenaton of collagen and to determine which methods are effective at significantly reducing pyrogens' levels present in raw collagen. To do so, raw collagen was subjected to vapor hydrogen peroxide (VHP), liquid hydrogen peroxide (LHP), dehydrothermal (DHT), chlorine dioxide, and nitrogen dioxide treatments.

To assess the effectiveness of the depyrogenation treatment methods, collagen samples were tested for percent reduction in pyrogen presence as compared to raw collagen pyrogen levels following particular treatment. DSC testing was also performed to determine the effect of each treatment on the transition temperature of the collagen. DSC testing was not conducted on the nitrogen dioxide samples.

### Example 1

### Comparing Depyrogenation Methods for Collagen

High pyrogen levels were present in batches of collagen purchased from Devro.

To evaluate various methods for depyrogenaton of collagen and to determine which methods are effective at significantly reducing pyrogens' levels present in raw collagen, raw collagen was subjected to vapor hydrogen peroxide (VHP), liquid hydrogen peroxide (LHP), dehydrothermal (DHT), chlorine dioxide, and nitrogen dioxide treatments. Raw, untreated collagen from Devro was used as control.

Samples varied in which depyrogenation method was used to treat the collagen.

### Vapor Hydrogen Peroxide (VHP) Treatment

Eight conditions of VHP, outlined below in Table 1, were tested to identify which VHP cycles would be effect at collagen depyrogenation. Four cycles were performed with two involving atmospheric processes (2 and 4 hour cycles at 800 ppm VHP) and two involving vacuum processes (cycles at 10 and 20 pulses each at 4 grams VHP per pulse). The samples were treated using, e.g., the Steris MD2000 deep vacuum sterilization system and the Steris VHPI000ED VHP generator with 30 ft³ isolator. The collagen samples were treated in Tyvec pouches for all of the VHP cycles.

The general VHP treatment process provided by Steris involves four major steps: dehumidification, conditioning, biodecontamination, and aeration. During a cycle, humidity was first removed using an integrated desiccant system followed by rapid injection of Vaprox (VHP sterilant) to condition the system and quickly raise the hydrogen peroxide level to a desired concentration. The VHP was maintained at the desired concentration for a set amount of time (Table 1) for the biodecontamination step and finally the vapor was broken down into safe byproducts, water vapor and oxygen, once the treatment process has been completed.

Four cycles were performed by Steris with two involving atmospheric processes (2 and 4 hour cycles at 800 ppm VHP) and two involving vacuum processes (cycles at 10 and 20 pulses each at 4 grams VHP per pulse). The collagen samples were treated in Tyvec pouches for all of the VHP cycles.

The cycles performed are outlined below in Table 1.

TABLE 1
VHP treated samples:

| Test Specimen | Duration | | Concentration | | Cycle Type |
|---|---|---|---|---|---|
| TS1 | 2 | Hr | 800 | ppm | Steris VHP1000ED with atmospheric pressure isolator |
| TS2 | 2 | Hr | 800 | ppm | Steris VHP1000ED with atmospheric pressure isolator |
| TS3 | 4 | Hr | 800 | ppm | Steris VHP1000ED with atmospheric pressure isolator |
| TS4 | 4 | Hr | 800 | ppm | Steris VHP1000ED with atmospheric pressure isolator |
| TS5 | 10 | pulses | 4 | g/pulse | Steris MD2000 deep vacuum sterilization system |
| TS6 | 10 | pulses | 4 | g/pulse | Steris MD2000 deep vacuum sterilization system |
| TS7 | 20 | pulses | 4 | g/pulse | Steris MD2000 deep vacuum sterilization system |
| TS8 | 20 | pulses | 4 | g/pulse | Steris MD2000 deep vacuum sterilization system |

The VHP treated collagen, the same as the original TS3 condition, was subjected to kinetic turbidimetric LAL testing.

### Liquid Hydrogen Peroxide (LHP)

Liquid hydrogen peroxide treatment was performed for collagen depyrogenation. Samples were prepared by soaking collagen in 500 mL of a commercially available 3% liquid hydrogen peroxide solution (CVS Pharmacy) for 2 hours. The collagen samples were then lyophilized to dry out collagen. The LHP treated collagen was subjected to kinetic chromogenic LAL testing.

### Dehydrothermal Treatment (DHT)

Dehydrothermal treatment (DHT) was performed for collagen depyrogenaton.

The DHT process was performed at a temperature of 105° C. and under a pressure of 150 mTorr for periods of 3, 6, 12, and 24 hours. The DHT samples were then subjected to kinetic turbidimetric LAL testing.

### Gaseous Chlorine Dioxide Treatment:

Gaseous chlorine dioxide was performed by ClorDiSys for collagen depyrogenation using an enclosed chlorine dioxide chamber at a dosage of 720 ppm per hour. The collagen samples were depyrogenated within Tyvek pouches and collagen samples exhibited a pink coloration following treatment to indicate exposure to the chlorine dioxide treatment process. The chlorine dioxide treatment process was not affected by temperature, produced no measurable residue, was non-carcinogenic, and was able to kill all viruses, bacteria, fungi and spores, and was able to completely fill all space contained in the chamber in order to evenly contact all surfaces.

ClO₂ sterilized samples were subjected to kinetic turbidimetric LAL testing.

### Nitrogen Dioxide Treatment

Nitrogen dioxide treatment for collagen depyrogenation (e.g., Noxilizer) was performed using an enclosed nitrogen dioxide treatment chamber. The collagen samples were treated in Tyvek pouches. A low humidity vacuum cycle with two pulses was selected for the collagen with a concentration of 10 mg/L of nitrogen dioxide, 40% relative humidity, and a 60 minute dwell time. The nitrogen dioxide treatment process resulted in slight yellowing/discoloration of the collagen.

NO₂ sterilized samples were subjected to kinetic turbidimetric LAL testing.

### Methods

LAL testing was performed with specifications outlined above.

The testing was performed at ambient temperature.

Only 1 sample was tested for pyrogen levels for the LHP treatment to obtain an initial assessment of the depyrogenation effectiveness.

At least 3 samples were tested for pyrogen levels for all other treatments to adequately assess the depyrogenation effectiveness of each process.

### Evaluation

For LAL testing, endotoxin presence was detected spectrophotometrically following extraction of sample previously incubated at 37° C.

Significant reduction in pyrogen levels as compared to the raw collagen samples was desired but no official acceptance criteria were established.

### Results

Table 2 below contains the average percent reductions in pyrogen presence as compared to the average raw collagen pyrogen level.

The VHP-TS3 method, VHP-TS4 method, all DHT methods and the ClO₂ depyrogenation method all resulted in tenfold reduction in pyrogen levels (seen in the percent reductions in Table 2) as compared to the raw collagen samples showing that these processes are highly effective at depyrogenation of bovine collagen.

LHP, NO₂, and the other VHP test methods were not as successful in the depyrogenation of the collagen samples.

TABLE 2
Average percent reduction of pyrogen level.

| | Average Percent Reduction |
|---|---|
| Sample | Compared to Raw Collagen |
| Description | Average |
| Raw Collagen | — |
| VHP - TS1 | No Change |
| VHP - TS2 | No Changes |
| VHP - TS3 | 89.84% |
| VHP - TS4 | 85.65% |
| VHP - TS5 | 53.18% |
| VHP - TS6 | 41.78% |
| VHP - TS7 | No Changes |
| VHP - TS8 | No Changes |
| LHP | No change |
| 3 hour DHT | 92.70% |
| 6 hour DHT | 93.75% |
| 12 hour DHT | 92.44% |
| 24 hour DHT | 95.99% |
| ClO₂ treated | 93.55% |
| NO₂ treated | No changes |

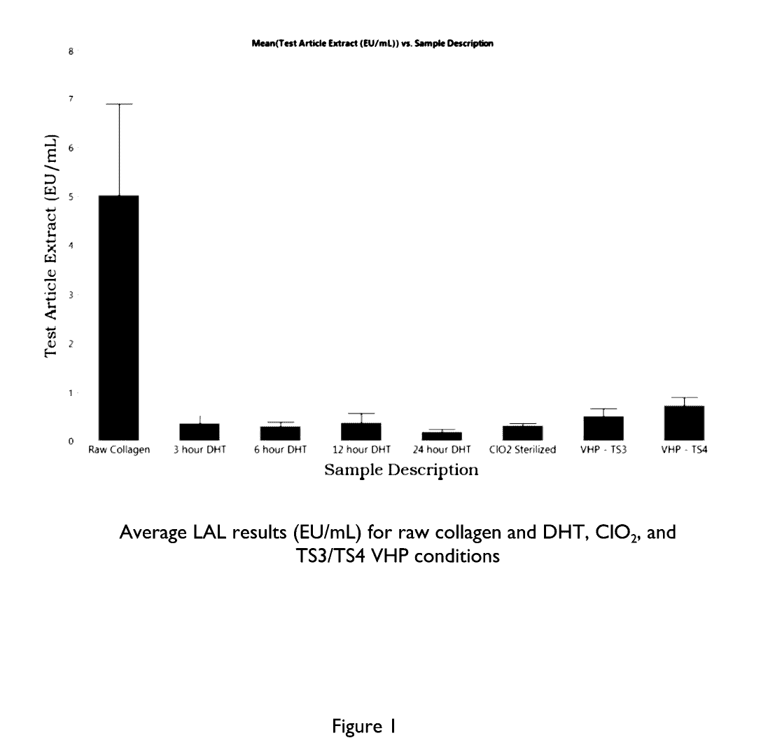

FIG. 1 show the average EU/mL endotoxin levels with standard deviation bars for each sample group tested. FIG. 1 shows the conditions with the highest reduction levels (DHT, VHP conditions TS3 and TS4, and the ClO₂ condition) as compared to the raw collagen.

Because of the tenfold (or higher) reduction in collagen pyrogen levels exhibited with the VHP-TS3 method, VHP-TS4 method, all DHT methods and the ClO₂ depyrogenation method treatment processes, these processes are deemed as being highly effective collagen depyrogenation methods.

### Example 2

DSC testing was performed to determine transition temperatures of the treated collagen samples and ensure that the treatment processes did not denature the collagen.

Design

The treated collagen samples were subjected to DSC testing in order to assess collagen transition temperatures.

Materials

| | | |
|---|---|---|
| i. | VHP treated collagen | CG-02-16, CG-02-27 |
| ii. | DHT treated collagen | CG-02-21, CG-02-22 |
| iii. | ClO₂ sterilized collagen | CG-02-26-1, CG-02-29-2 |
| iv. | High purity water (USP Type 1) | |
| v. | DSC | |
| vi. | DSC sample pans | |

Processing Methods

Samples were tested and the resulting DSC data was analyzed.

Sample Variation

Samples varied in which depyrogenation method was used to treat the collagen.

Conditions

DSC scans were conducted in the R&D Laboratory under ambient conditions defined as 20-25 degrees Celsius and 40-60% Relative Humidity.

Parameter Selection

DSC test parameters
1. 70° C. was selected as the maximum to fully evaluate the transition temperature of collagen in water.
2. 20° C. was the temperature at which the colorimeter began recording data.
3. The temperature was increased at a rate of 5° C. per minute

Three replicates were evaluated for each DSC test. Only sample sets with high pyrogen level reduction in comparison to the raw collagen were tested (CG-02-16 (TS3, TS4), CG-02-21-2, CG-02-21-5, and CG-02-29-2). Note that the 3 hour (worst case depyrogenation scenario) and 24 hour (best case depyrogenation scenario) DHT samples were subjected to DSC testing.

Test Completion

DSC testing was completed once 70° C. was reached.

Results

Table 3 below shows the average DSC values for each condition.

TABLE 3
Average DSC Values

| Sample | Average Transition |
|---|---|
| Description | Temperature (° C.) |
| Raw Collagen | 54.60 |
| VHP - TS3 | 55.03 |
| VHP - TS4 | 54.99 |
| 3 hour DHT | 52.69 |
| 24 hour DHT | 48.93 |
| ClO₂ Treated | 54.35 |

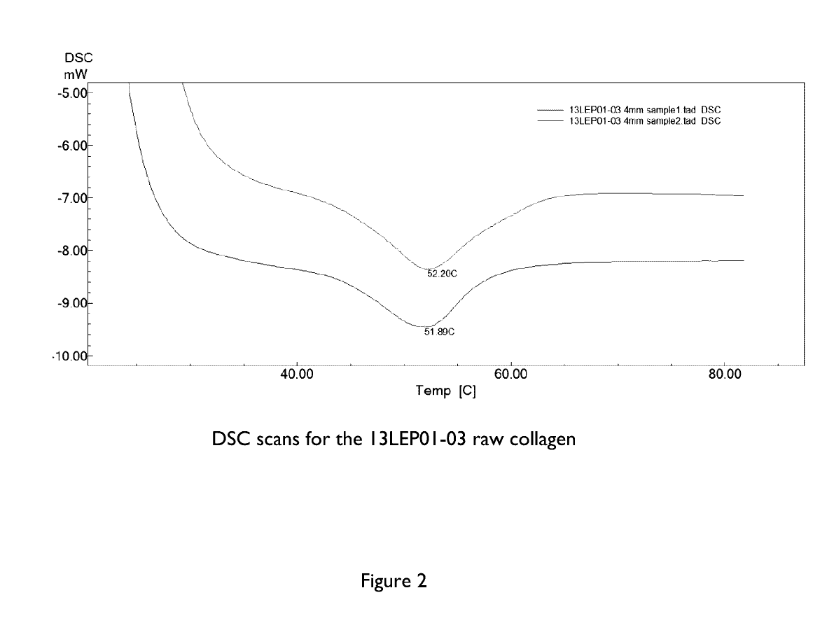

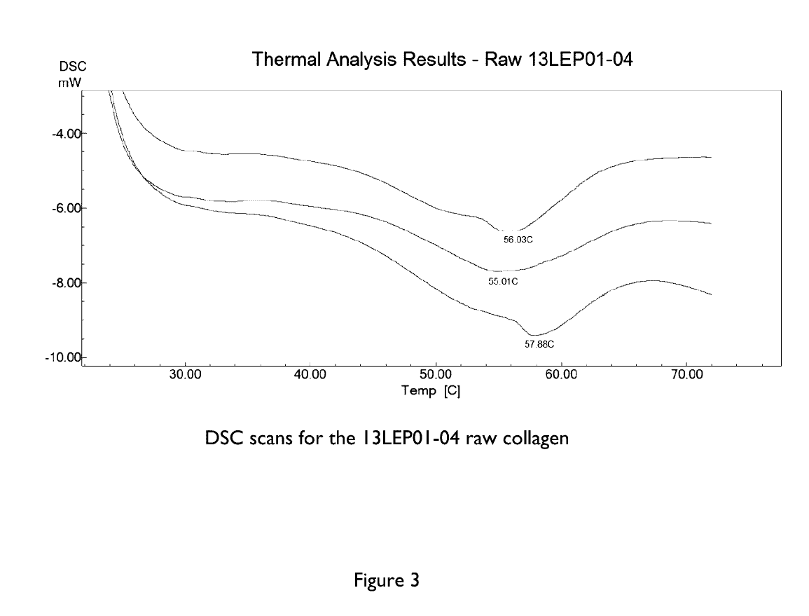

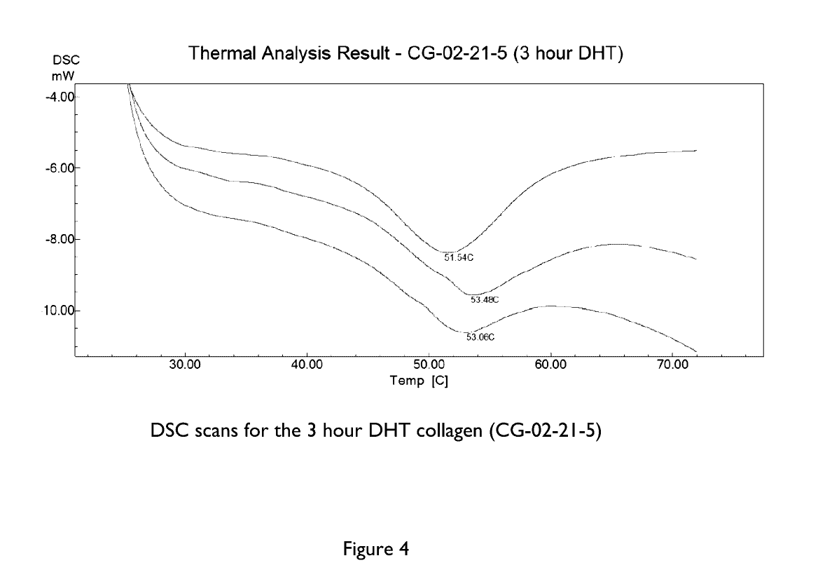

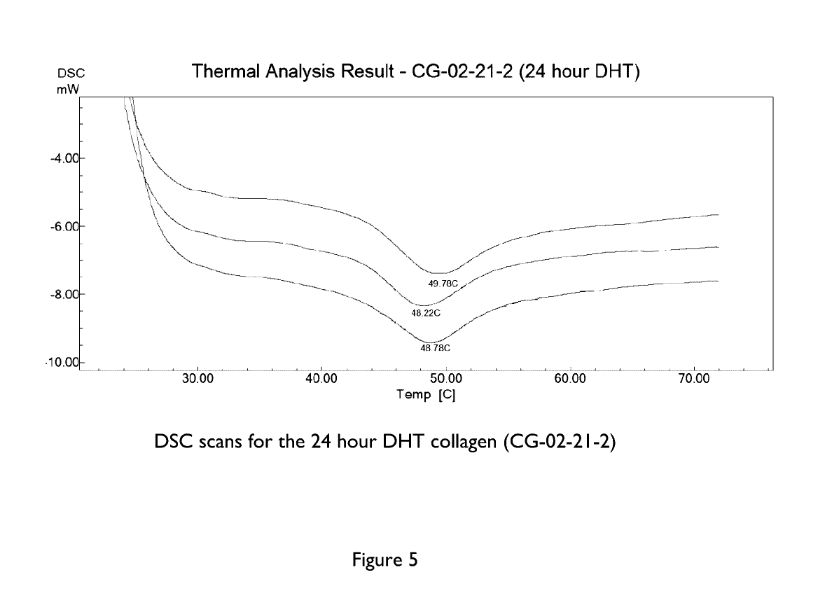

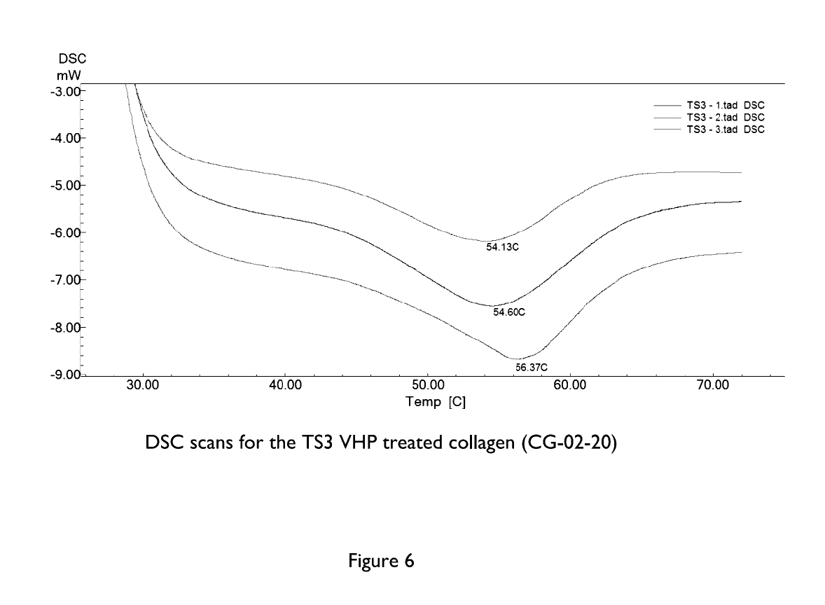

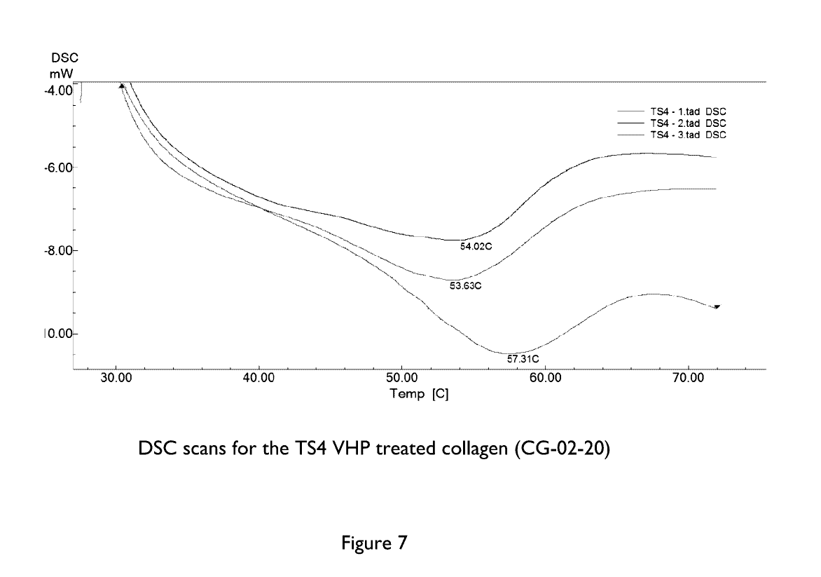

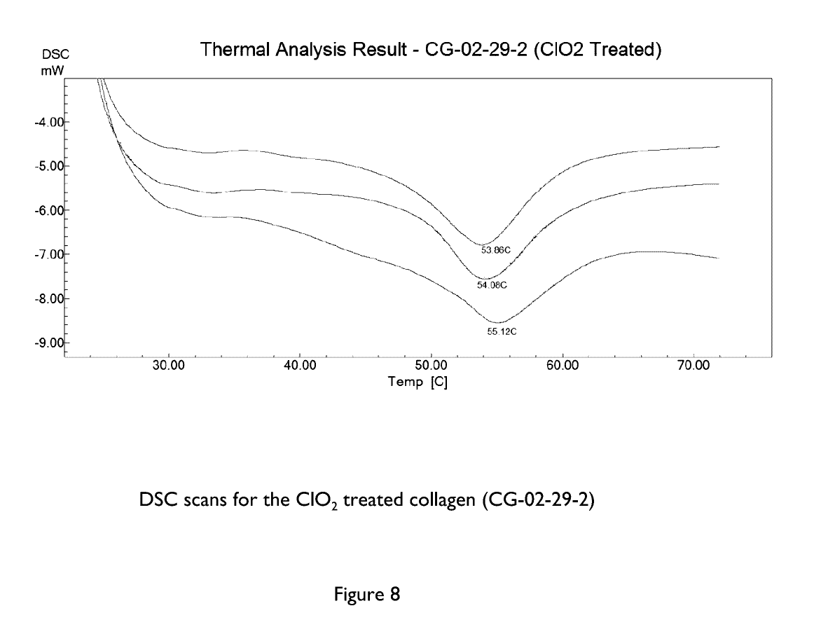

FIGS. 2 through 8 show the DSC scans for each condition tested (raw collagen, VHP-TS3, VHP-TS4, 3 hour DHT, 24 hour DHT, ClO₂ treated).

Discussion

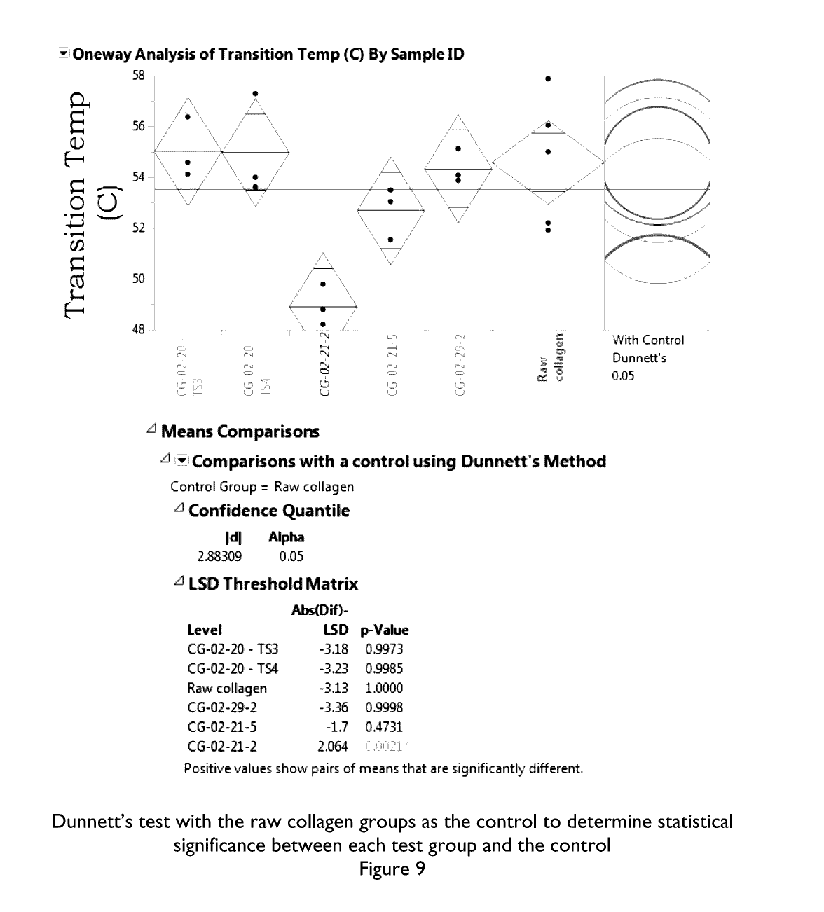

Referring to the Dunnett's test shown in FIG. 9, with the exception of the 24 hour DHT condition, all other collagen treatment conditions did not significantly change the collagen transition temperature. Because of this, these conditions (VHP, ClO₂, and 3 hour DHT) resulted in collagen structures not significantly different from the initial raw collagen.

These findings are important in that they signify that the VHP, 3 hour DHT, and ClO₂ treatment conditions not only depyrogenate collagen but also do not significantly denature the collagen during the treatment processes.

### Example 3

Circular dichroism (CD) analysis (using Jasco J-815 Circular Dichroism Spectrometer) was performed to assess the collagen structure following one of several protein depyrogenation methods. Devro collagen was subjected to either vapor hydrogen peroxide (VHP), dehydrothermal (DHT), or chlorine dioxide treatment; <4 mm Devro collagen was subjected to VHP and ClO₂ and <6 mm Devro collagen was subjected to a three hour DHT cycle. Using CD, the collagen structures were compared before and after depyrogenation to assess the degree of protein structural change following treatment.

The percent α-helix, 3₁₀ helix, β-sheet, turn, Polyproline-II helix, and unordered structures for each of the collagen samples was determined using CD. Comparing untreated <4 mm Devro collagen to <4 mm Devro treated either with VHP or ClO₂, treatment resulted in minor increases in % α-helix structure and minor decreases in % unordered structure; the other protein structures exhibited virtually no change.

Test and Control Samples

Control and test sample selection criteria:

Control samples consisted of Devro collagen samples that were not subjected to any depyrogenation treatment:

Control sample 1: <4 mm Devro collagen

Control sample 2: <6 mm Devro collagen

Test samples consisted Devro collagen subjected to one of several depyrogenation treatments (each sample varied in method used for depyrogenation):

Test sample: <4 mm Devro collagen treated with ClO₂

Test sample: <4 mm Devro collagen treated with VHP

Test sample: <6 mm Devro collagen treated with a three hour DHT cycle

Processing Methods

Each sample was digested using pepsin enzyme. Specifically, 20 mM Acetic Acid was prepared from glacial Acetic Acid.

Next 0.1% and 0.4% pepsin digest solution was prepared in 20 mM acetic acid.

Next the collagen standard (Advanced Biomatrix Pure-Col #5015-A) was digested with 0.1% pepsin (1:1, V:V) for two hours at room temperature. 50 mg of each solid collagen test article was weighed and the samples were shredded thoroughly using a scalpel/tweezers to increase available surface area for pepsin digestion. 5 ml 0.1% pepsin was added to the collagen samples. Samples were then incubated for 1 hour at 37° C. Once the incubation period was complete, the collagen standard and test articles were kept at 4° C. to deactivate pepsin enzymatic activity. The samples were centrifuged at 2000 RPM for 3 minutes to remove undigested collagen from the supernatant.

Next the protein content of the supernatant collagen test articles was determined by using the Pierce BCA Protein Assay Kit.

Once collagen samples have been digested as described above and the protein concentrations have been determined, the samples were prepared for CD analysis. CD analysis was performed using the Jasco 815 CD Spectrometer to analyze the protein structures of collagen samples. The collagen test articles were diluted to a concentration between 0.2 and 1.0 mmol/L and placed in the 1 mm path cell. The CD spectra of all test articles were collected at 25° C. with three accumulations.

To analyze the secondary structure of the protein following collection of the CD spectra, the CDPro Analysis performed. The cell length and sample concentration (the sample concentration is determined from the Mean Residue Weight, which estimated to be 110 for proteins like collagen) were entered. The CDSSTR method was used with reference spectra SP22X to analyze the test articles. The % α-helix, %3/10 helix, %β sheet, % turn, % polyproline II, % unordered data were recorded; these percentages provide a quantitative indication of the structures present in the collagen.

Methods

The digested collagen samples were diluted using 20 mM acetic acid in order to formulated samples with protein concentrations acceptable for CD analysis.

For each sample set, n=1 CD scans were obtained to assess the collagen structures before and after depyrogenation treatments.

Testing was completed once CD scans has been obtained for each collagen sample.

Acceptance Criteria

The collagen structure should not be altered due to the depyrogenation treatments but no official acceptance criteria were established.

Results

Experimental Data

Table 4 below shows the percentage of each present collagen structure, as determined through CD analysis.

TABLE 4
Percent collagen structures

| Structure/Sample | % A- | % 3/10 | % B | | | |
|---|---|---|---|---|---|---|
| (@25 C. PreThermal) | Helix | Helix | Sheet | % Turn | % Polyproline II | % Unordered |
| Raw 4 mm | 2.2 | 13.7 | 13.1 | 21.4 | 17.9 | 31.7 |
| VHP 4 mm | 3.4 | 15.1 | 11.7 | 22.3 | 18 | 29.8 |
| ClO2 4 mm | 3.8 | 14.4 | 12.1 | 21.3 | 18.5 | 29.9 |
| Raw 6 mm | 1.2 | 14 | 8.2 | 24.3 | 16 | 36 |
| 3 h DHT 6 mm | 1.7 | 13.5 | 12.2 | 22.2 | 16.3 | 34.2 |

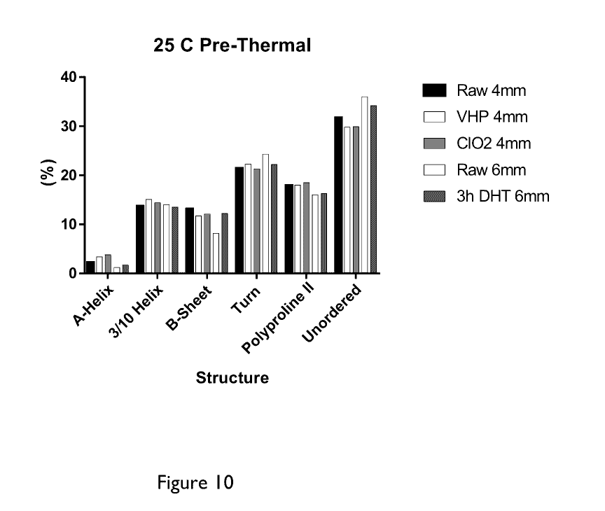

FIG. 10 displays a graphical comparison of each of the percent collagen structures for each of the five collagen samples. As seen in FIG. 10, the depyrogenation treatments have very little effect in terms of altering the protein structures of the collagen samples. Comparing untreated <4 mm Devro collagen to <4 mm Devro treated either with VHP or ClO2, treatment resulted in minor increases in % α-helix structure and minor decreases in % unordered structure; the other protein structures exhibited virtually no change. Comparing untreated <6 mm Devro collagen to <6 mm Devro treated with a three hour DHT cycle, a 4% increase in % β-sheet structure was the only noticeable change due to treatment.

### CONCLUSIONS

The percent α-helix, 310 helix, β-sheet, turn, Polyproline-II helix, and unordered structures for each of the collagen samples was determined using CD spectrometry. Comparing untreated <4 mm Devro collagen to <4 mm Devro treated either with VHP or ClO2, treatment resulted in minor increases in % α-helix structure and minor decreases in % unordered structure; the other protein structures exhibited virtually no change. Comparing untreated <6 mm Devro collagen to <6 mm Devro treated with a three hour DHT cycle, a 4% increase in % β-sheet structure was the only noticeable change due to treatment. Overall, the <4 mm and <6 mm Devro structure collagen is not altered as a result of VHP, ClO2, and DHT depyrogenation processes.

The results from this experiment give indications as to whether or not VHP, ClO₂, and DHT depyrogenation methods alter the protein structure of Devro collagen. Overall, the <4 mm and <6 mm Devro collagen structure is not altered as a result of VHP, ClO₂, and DHT depyrogenation processes.

It is clear that the methods for depyrogenation and reduction of endotoxins have wide applicability to the field and profession of medicine, and most particularly to medical devices that utilize protein.

It should be understood that various modifications within the scope of this invention can be made by one of ordinary skill in the art without departing from the spirit thereof and without undue experimentation. For example, the cards can have a wide range of shapes, including a cut-out along one margin to provide a carry handle, to provide the functionalities disclosed herein. This invention is therefore to be defined by the scope of the appended claims as broadly as the prior art will permit, and in view of the specification if need be, including a full range of current and future equivalents thereof.

1. A method for depyrogenaton of protein, comprising:
exposing the protein to vapor hydrogen peroxide for a duration of time and at a concentration of vapor hydrogen peroxide sufficient to reduce pyrogens,
wherein the exposing step does not substantially change the tertiary structure of the protein and does not denature the protein.

2. The method of claim 1, wherein the concentration of hydrogen peroxide is in a range from about 200 ppm to about 2000 ppm hydrogen peroxide in an atmospheric pressure isolator, and the duration of the exposing step ranges from about 1 hours to about 48 hours.

3. The method of claim 1, wherein the concentration of hydrogen peroxide is about 800 ppm hydrogen peroxide in an atmospheric pressure isolator, and the duration of the exposing step is about 4 hours.

4. The method of claim 1, further comprising aerating the protein.

5. The method of claim 1, wherein the protein is collagen, fibronectin, vitronectin, laminin, pectin, elastin, osteopontin, bone sialoprotein, thrombospondin, fibrinogen, gelatin, or combinations thereof.

6. A method for depyrogenaton of protein, comprising
exposing the protein to gaseous chloride dioxide for a duration of time and at a concentration of gaseous chloride dioxide sufficient to reduce pyrogens,
wherein the exposing step does not substantially change the tertiary structure of the protein and does not denature of the protein.

7. The method of claim 6, wherein the concentration of gaseous chloride dioxide is in a range from about 100 ppm to about 2000 ppm gaseous chloride dioxide per hour in an atmospheric pressure isolator.

8. The method of claim 6, wherein the concentration of gaseous chloride dioxide is about 720 ppm gaseous chloride dioxide per hour in an atmospheric pressure isolator.

9. The method of claim 6, wherein the protein is collagen, fibronectin, vitronectin, laminin, pectin, elastin, osteopontin, bone sialoprotein, thrombospondin, fibrinogen, gelatin, or combinations thereof.

10. A method for depyrogenaton of protein comprising exposing the protein to a dehydrothermal treatment (DHT) for a duration of time sufficient to reduce pyrogens, wherein the exposing step does not substantially change the tertiary structure of the protein and does not denature of the protein.

11. The method of claim 10, wherein the exposing step is at a temperature ranging from about 60° C. to about 130° C. and under a pressure of from about 10 mTorr to about 1000 mTorr.

12. The method of claim 10, wherein the exposing step is at a temperature of about 105° C. and under a pressure of 150 mTorr.

13. The method of claim 10, wherein the duration of time sufficient to reduce pyrogens is from about 1 hour to about 48 hours.

14. The method of claim 10, wherein the protein is collagen, fibronectin, vitronectin, laminin, pectin, elastin, osteopontin, bone sialoprotein, thrombospondin, fibrinogen, gelatin, or combinations thereof.

15. A composition comprising collagen with substantially no amount of pyrogens, substantially no change in the tertiary structure of collagen, and substantially no denaturing of collagen.

16. The composition of claim 15, wherein the composition is suitable for wound care, hemostasis, duraplasty and as an adhesion barrier.

17. The composition of claim 16, further comprising a ceramic material.

18. The composition of claim 17, wherein the ceramic material is selected from the group consisting of bioactive glass, tricalcium phosphate (TCP), hydroxyapatite calcium sulfate.

19. The composition of claim 18, wherein the bioactive glass is selected from the group consisting of a silicate bioactive glass, a borate bioactive glass, titanate bioactive glass, and zirconate bioactive glass.

20. The composition of claim 19, wherein the bioactive glass is in the shape of fibers, spheres, particles, or a combination thereof.

21. The composition of claim 18, wherein the bioactive glass is melt-derived or sol-gel derived.

22. The composition of claim 18, further comprising at least one of glycosaminoglycan, a pharmaceutical agent, or a protein.

23. The composition of claim 18 for use in regenerating bone at or near the site of a bony defect.
